(19) United States
(12) Patent Application Publication
(10) Pub. No.: US 2009/0142275 A1
(43) Pub. Date: Jun. 4, 2009

(54) Wound Suture Capable of Identifying the Presence of Bacteria

(75) Inventors: Erica M. Phillips, Woodstock, GA (US); J. Gavin MacDonald, Decatur, GA (US); Stephanie M. Martin, Woodstock, GA (US)

Correspondence Address: DORITY & MANNING, P.A., POST OFFICE BOX 1449, GREENVILLE, SC, 29602-1449, US

(73) Assignee: KIMBERLY-CLARK WORLDWIDE, INC., Neenah, WI (US)

(21) Appl. No.: 11/947,296

(22) Filed: Nov. 29, 2007

(51) Int. Cl. A61L 17/04; A61B 5/00; A61K 49/00

(52) U.S. Cl. 424/9.8; 424/9.1; 606/139

(57) ABSTRACT

A wound suture containing a solvatochromatic indicator that undergoes a color change in the presence of bacteria often associated with surgical site infection is provided. Such a color change provides a “real time” indication of the onset of infection, which may alert medical staff to apply an appropriate antimicrobial treatment (e.g., antibiotic) to the patient (e.g., human or animal) before a more serious infection occurs. The patient may also be able to accurately monitor the condition of a wound after discharge from the hospital. Further, the lack of a color change may provide the medical staff or patient with the assurance that the area is generally free of infection and clean.

### BACKGROUND OF THE INVENTION

Surgical site infections are one of the most common cause of nosocomial, i.e., hospital-acquired, infections and are typically due to bacterial species, such as Streptococcus pyogenes (S. pyogenes), Pseudomonas aeruginosa (P. aeruginosa), Enterococcus faecalis (E. faecalis), Proteus mirabilis (P. mirabilis), Serratia marcescens (S. marcescens), Enterobacter clocae (E. clocae), Acetinobacter anitratus (A. anitratus), Klebsiella pneumoniae (K. pneumonia), Escherichia coli (E. coli), Staphyloccus aureus (S. aureus), coagulase-negative Staphylococci, and Enterococcus spp. It has been found that sutures for wound sites actually contribute to surgical site infection via suture canal, perisutural cuff of dead epidermis, dermis and subcutaneous fat. Namely, sutures can provide initiate infection by (1) providing a route of entry from the skin to subcutaneous tissue, (2) providing a route of entry from intradermal structures, hair follicles, sebaceous glands etc, (3) maintaining patency of tract for 5-10 days, (4) causing foreign body reaction with associated local tissue autolysis, so that sutures can break down the tissue barrier into more infected intradermal structures (e.g., sebaceous gland) which were not open at the time of initial passage of suture and (5) foreign body reaction and local tissue autolysis. Non-suture Closure of Wound Using Cyanoacrylate; Dalvi, et al.; Vol. 32, Issue 2, pp. 97-100 (1986).

The most common way of preventing infection is to administer prophylactic antibiotic drugs. While generally effective, this strategy has the unintended effect of breeding resistant strains of bacteria. Rather than using routine prophylaxis, a better approach is to practice good wound management, i.e., keep the area free from bacteria before, during, and after surgery, and carefully monitor the site for infection during healing. Normal monitoring methods include close observation of the wound site for slow healing, signs of inflammation and pus, as well as measuring the patient's temperature for signs of fever. Unfortunately, many symptoms are only evident after the infection is already established. Furthermore, after a patient is discharged from the hospital, they become responsible for monitoring their own healthcare, and the symptoms of infection may not be evident to the unskilled patient.

In response to these problems, techniques have been developed for detecting wound-specific microorganisms. One such technique is described, for instance, U.S. Patent Application Publication No. 2005/0142622 to Sanders, et al. The technique of Sanders, et al. involves contacting a sample with a labeled substrate for an enzyme produced or secreted by a microorganism, and thereafter detecting the modification of the substrate. Unfortunately, however, such techniques are far too complex for practical use. Instead, what is needed is a relatively simple and effective technique for detecting the early stages of bacterial infection.

### SUMMARY OF THE INVENTION

In accordance with one embodiment of the present invention, a wound suture is disclosed that comprises at least one filament. The filament contains a solvatochromatic indicator that undergoes a detectable color change in the presence of bacteria. In accordance with another embodiment of the present invention, a method for detecting the presence of bacteria at a surgical incision site is disclosed. The method comprises passing a needled suture through tissue to create a wound closure, the suture containing a solvatochromatic indicator that undergoes a detectable color change in the presence of bacteria. Thereafter, the suture is observed for the color change.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figure in which:

FIG. 1 is a schematic illustration of one embodiment of a suture-needle assembly that may be employed in the present invention.

Repeat use of reference characters in the present specification and drawing is intended to represent same or analogous features or elements of the invention.

### DETAILED DESCRIPTION OF REPRESENTATIVE EMBODIMENTS

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

Generally speaking, the present invention is directed to a wound suture containing a solvatochromatic indicator that undergoes a color change in the presence of bacteria often associated with surgical site infection, such as Streptococcus pyogenes (S. pyogenes), Pseudomonas aeruginosa (P. aeruginosa), Enterococcus faecalis (E. faecalis), Proteus mirabilis (P. mirabilis), Serratia marcescens (S. marcescens), Enterobacter clocae (E. clocae), Acetinobacter anitratus (A. anitratus), Klebsiella pneumoniae (K. pneumonia), Escherichia coli (E. coli), Staphyloccus aureus (S. aureus), coagulase-negative Staphylococci, Enterococcus spp., and so forth. Such a color change provides a “real time” indication of the onset of infection, which may alert medical staff to apply an appropriate antimicrobial treatment (e.g., antibiotic) to the patient (e.g., human or animal) before a more serious infection occurs. The patient may also be able to accurately monitor the condition of a wound after discharge from the hospital. Further, the lack of a color change may provide the medical staff or patient with the assurance that the area is generally free of infection and clean.

Merocyanine indicators (e.g., mono-, di-, and tri-merocyanines) are one example of a type of solvatochromatic indicator that may be employed in the present invention. Merocyanine indicators, such as merocyanine 540, fall within the donor—simple acceptor indicator classification of Griffiths as discussed in “Colour and Constitution of Organic Molecules” Academic Press, London (1976). More specifically, merocyanine indicators have a basic nucleus and acidic nucleus separated by a conjugated chain having an even number of methine carbons. Such indicators possess a carbonyl group that acts as an electron acceptor moiety. The electron acceptor is conjugated to an electron donating group, such as a hydroxyl or amino group. The merocyanine indicators may be cyclic or acyclic (e.g., vinylalogous amides of cyclic merocyanine indicators). For example, cyclic merocyanine indicators generally have the following structure:

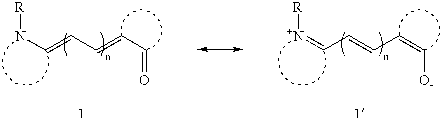

wherein, n is any integer, including 0. As indicated above by the general structures 1 and 1′, merocyanine indicators typically have a charge separated (i.e., “zwitterionic”) resonance form. Zwitterionic indicators are those that contain both positive and negative charges and are net neutral, but highly charged. Without intending to be limited by theory, it is believed that the zwitterionic form contributes significantly to the ground state of the indicator. The color produced by such indicators thus depends on the molecular polarity difference between the ground and excited state of the indicator. One particular example of a merocyanine indicator that has a ground state more polar than the excited state is set forth below as structure 2.

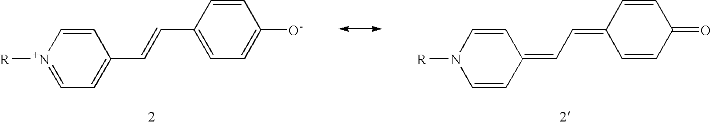

The charge-separated left hand canonical 2 is a major contributor to the ground state whereas the right hand canonical 2′ is a major contributor to the first excited state. Still other examples of suitable merocyanine indicators are set forth below in the following structures 3-13.

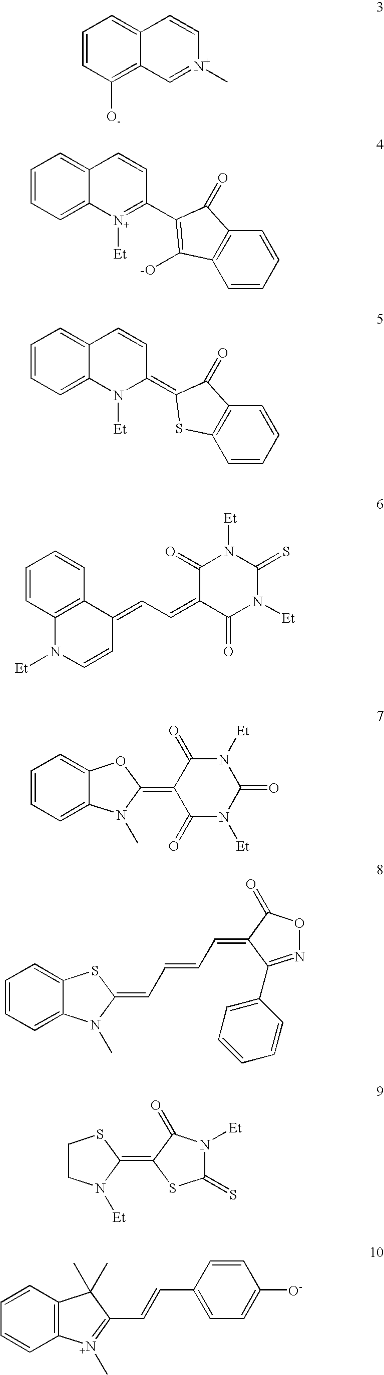

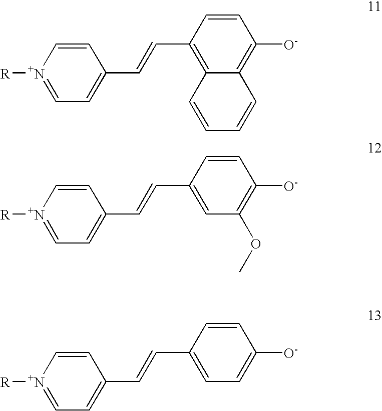

wherein, “R” is a group, such as methyl, alkyl, aryl, phenyl, etc.

Indigo is another example of a suitable solvatochromatic indicator for use in the present invention. Indigo has a ground state that is significantly less polar than the excited state. For example, indigo generally has the following structure 14:

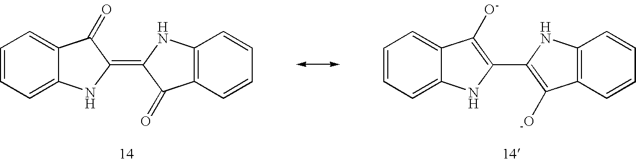

The left hand canonical form 14 is a major contributor to the ground state of the indicator, whereas the right hand canonical 14′ is a major contributor to the excited state.

Other suitable solvatochromatic indicators that may be used in the present invention include those that possess a permanent zwitterionic form. That is, these indicators have formal positive and negative charges contained within a contiguous π-electron system. Contrary to the merocyanine indicators referenced above, a neutral resonance structure cannot be drawn for such permanent zwitterionic indicators. Exemplary indicators of this class include N-phenolate betaine indicators, such as those having the following general structure:

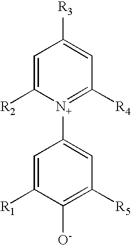

wherein R₁—R₅ are independently selected from the group consisting of hydrogen, a nitro group (e.g., nitrogen), a halogen, or a linear, branched, or cyclic C₁ to C₂₀ group (e.g., alkyl, phenyl, aryl, pyridinyl, etc.), which may be saturated or unsaturated and unsubstituted or optionally substituted at the same or at different carbon atoms with one, two or more halogen, nitro, cyano, hydroxy, alkoxy, amino, phenyl, aryl, pyridinyl, or alkylamino groups. For example, the N-phenolate betaine indicator may be 4-(2,4,6-triphenylpyridinium-1-yl)-2,6-diphenylphenolate (Reichardt's dye) having the following general structure 15:

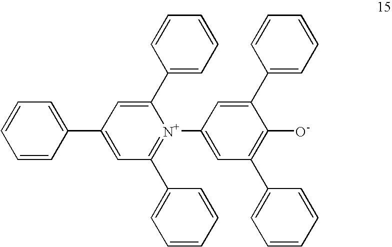

Reichardt's dye shows strong negative solvatochromism and may thus undergo a significant color change from blue to colorless in the presence of bacteria. That is, Reichardt's dye displays a shift in absorbance to a shorter wavelength and thus has visible color changes as solvent eluent strength (polarity) increases. Still other examples of suitable negatively solvatochromatic pyridinium N-phenolate betaine indicators are set forth below in structures 16-23:

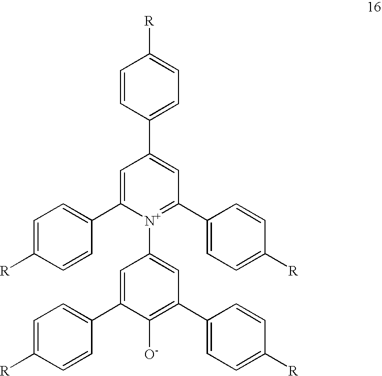

wherein, R is hydrogen, —C(CH₃)₃, —CF₃, or C₆F₁₃.

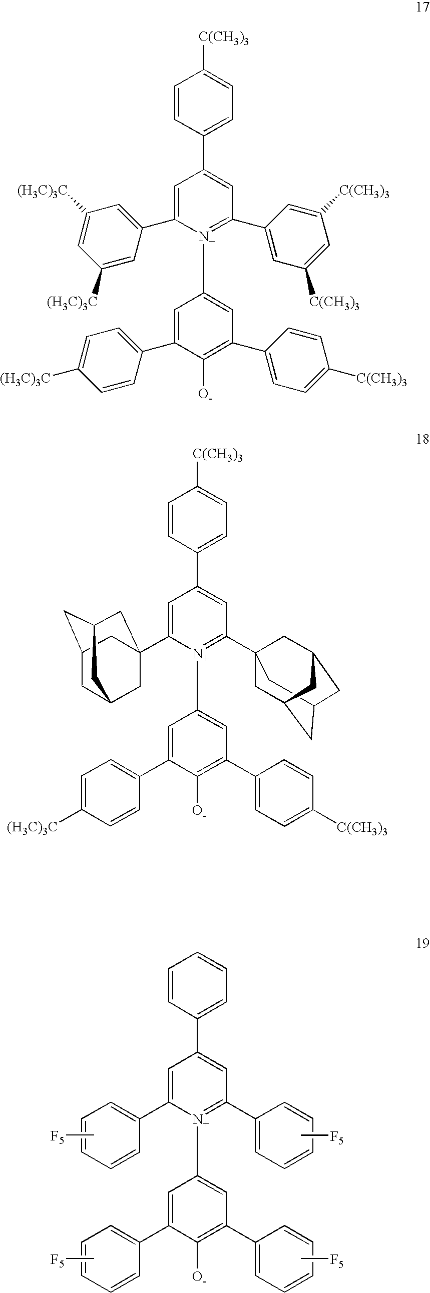

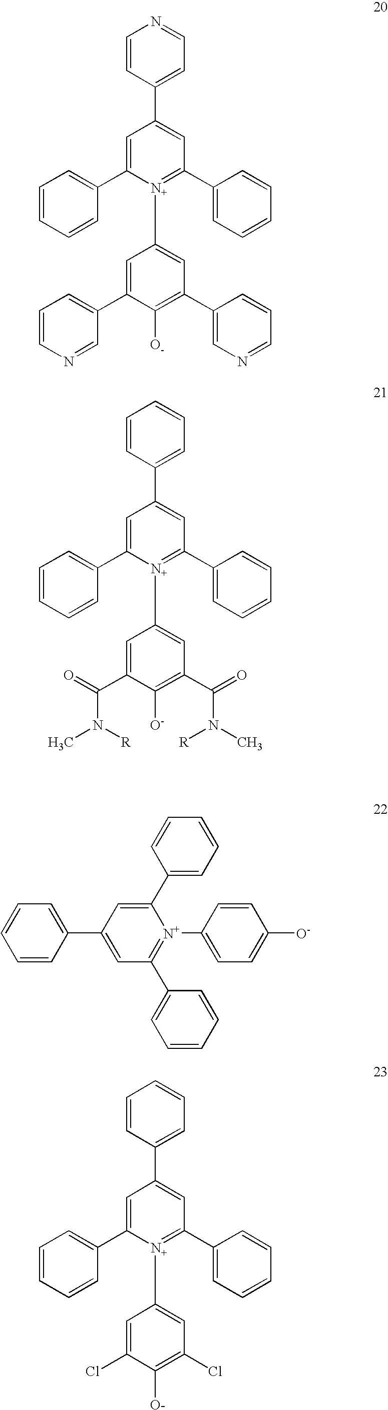

Still additional examples of indicators having a permanent zwitterionic form include indicators having the following general structure 24:

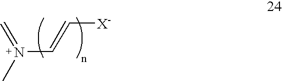

wherein, n is 0 or greater, and X is oxygen, carbon, nitrogen, sulfur, etc. Particular examples of the permanent zwitterionic indicator shown in structure 24 include the following structures 25-33.

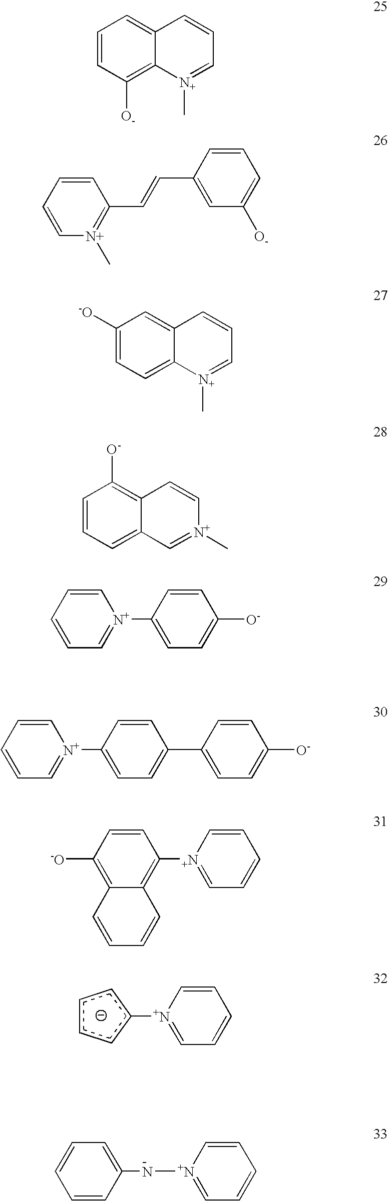

Still other suitable solvatochromatic indicators may include, but are not limited to 4-dicyanmethylene-2-methyl-6-(p-dimethylaminostyryl)-4H-pyran (DCM); 6-propionyl-2-(dimethylamino)naphthalene (PRODAN); 9-(diethylamino)-5H-benzo[a]phenox-azin-5-one (Nile Red); 4-(dicyanovinyl)julolidine (DCVJ); phenol blue; stilbazolium indicators; coumarin indicators; ketocyanine indicators; N,N-dimethyl-4-nitroaniline (NDMNA) and N-methyl-2-nitroaniline (NM2NA); Nile blue; 1-anilinonaphthalene-8-sulfonic acid (1,8-ANS), and dapoxylbutylsulfonamide (DBS) and other dapoxyl analogs. Besides the above-mentioned indicators, still other suitable indicators that may be used in the present invention include, but are not limited to, 4-[2-N-substituted-1,4-hydropyridin-4-ylidine)ethylidene]cyclohexa-2,5-dien-1-one, red pyrazolone indicators, azomethine indicators, indoaniline indicators, and mixtures thereof.

Although the above-referenced indicators are classified as solvatochromic, it should be understood that the present invention is not necessarily limited to any particular mechanism for the color change of the indicator. Even when a solvatochromic indicator is employed, other mechanisms may actually be wholly or partially responsible for the color change of the indicator. For example, acid-base or proton donation reactions between the indicator and microbe may result in the color change. As an example, highly organized acid moieties on bacteria cell walls may protonate certain indicators, resulting in a loss of color. Redox reactions between the indicator and microbe may likewise contribute to the color change.

Regardless of the type employed, the indicator is applied to a wound suture that is subsequently used to close a wound (e.g., surgical incision site). Suitable wound sutures may include both absorbable and non-absorbable, yet biostable sutures. Absorbable sutures, for example, may be formed from absorbable synthetic polymers, such as aliphatic polyesters, aromatic polyesters, aliphatic-aromatic copolyesters, polyesteramides; polycarbonates, polyanhydrides, polysaccharides, and so forth. Particularly suitable absorbable synthetic polymers may include polycaprolactone, polyesteramides, modified polyethylene terephthalate, polylactic acid and its copolymers, terpolymers based on polylactic acid, polyglycolic acid, polyalkylene carbonates (e.g., polyethylene carbonate, poly(glycolide-co-trimethylene carbonate, etc.), polyhydroxyalkanoates, poly-3-hydroxybutyrate, poly-3-hydroxyvalerate, poly-3-hydroxybutyrate-co-4-hydroybutyrate, poly-3-hydroxybutyrate-co-3-hydroxyvalerate copolymers, poly-3-hydroxybutyrate-co-3-hydroxyhexanoate, poly-3-hydroxybutyrate-co-3-hydroxyoctanoate, poly-3-hydroxybutyrate-co-3-hydroxydecanoate, poly-3-hydroxybutyrate-co-3-hydroxyoctadecanoate, and succinate-based aliphatic polymers (e.g., polybutylene succinate, polybutylene succinate adipate, polyethylene succinate, etc.). Natural absorbable polymers, such as collagen or surgical gut (e.g., chromic or fast absorbing), may also be employed. Examples of suitable non-absorbable, yet biostable materials may include polypropylene, nylon, polyethylene, polyesters (e.g., polyethylene terephthalate), silk, cotton, carbon, steel, and so forth.

The construction of the wound suture may be monofilament or multifilament, such as a bundle of individual filaments, a yarn or tow (that may be entangled, twisted or plied) or filaments or yarns that have been braided, knitted or woven. Suitable braided multifilament constructions for sutures are described in U.S. Pat. Nos. 5,019,093; 5,059,213 and 4,959,069, which are incorporated herein in the entirety by reference thereto for all purposes. Typically, at least one of the filaments in a multifilament core is oriented so that a significant number of the molecules within the fiber are positioned substantially parallel to the length of the fiber to impart strength parallel to the fibers length. The filaments are generally drawn at least 2 times their original length to orient the molecules in the fibers. Still other known suture constructions may be employed, such as surgical meshes (e.g., hernia repair mesh), brachy seed spacers, etc.

If desired, the indicator may be incorporated into the wound suture during its formation. Alternatively, the indicator may be coated onto all or only a portion of the wound suture. Suitable techniques for coating the indicator onto the suture include printing, dipping, spraying, melt extruding, coating (e.g., solvent coating, powder coating, brush coating, etc.), and so forth. In one embodiment, for example, the wound suture is dipped into a solution containing the indicator. Besides the indicator, the solution may contain other components, such as a mobile carrier. The carrier may be a liquid, gas, gel, etc., and may be selected to provide the desired performance (time for change of color, contrast between different areas, and sensitivity) of the indicator. In some embodiments, for instance, the carrier may be an aqueous solvent, such as water, as well as a non-aqueous solvent, such as glycols (e.g., propylene glycol, butylene glycol, triethylene glycol, hexylene glycol, polyethylene glycols, ethoxydiglycol, and dipropyleneglycol); alcohols (e.g., methanol, ethanol, n-propanol, and isopropanol); triglycerides; ethyl acetate; acetone; triacetin; acetonitrile, tetrahydrafuran; xylenes; formaldehydes (e.g., dimethylformamide, “DMF”); etc. Upon application, the solution may be dried to remove the carrier and leave a residue of the indicator for interacting with a microorganism.

Other additives may also be employed, either separately or in conjunction with the indicator. In one embodiment, for instance, cyclodextrins are employed that enhance the sensitivity and contrast of an indicator. While not wishing to be bound by theory, such additives may inhibit the crystallization of the indicator and thus provide a more vivid color and also enhance detection sensitivity. That is, single indicator molecules have greater sensitivity for microorganisms because each indicator molecule is free to interact with the microbial membrane. In contrast, small crystals of indicator have to first dissolve and then penetrate the membrane. Examples of suitable cyclodextrins may include, but are not limited to, hydroxypropyl-β-cyclodextrin, hydroxyethyl-β-cyclodextrin, γ-cyclodextrin, hydroxypropyl-γ-cyclodextrin, and hydroxyethyl-γ-cyclodextrin, which are commercially available from Cerestar International of Hammond, Ind.

Surfactants may also help enhance the sensitivity and contrast provided by the indicator. Particularly desired surfactants are nonionic surfactants, such as ethoxylated alkylphenols, ethoxylated and propoxylated fatty alcohols, ethylene oxide-propylene oxide block copolymers, ethoxylated esters of fatty (C₈-C₁₈) acids, condensation products of ethylene oxide with long chain amines or amides, condensation products of ethylene oxide with alcohols, acetylenic diols, and mixtures thereof. Various specific examples of suitable nonionic surfactants include, but are not limited to, methyl gluceth-10, PEG-20 methyl glucose distearate, PEG-20 methyl glucose sesquistearate, C₁₁₋₁₅ pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C₆-C₂₂) alcohol, including 3 to 20 ethylene oxide moieties, polyoxyethylene-20 isohexadecyl ether, polyoxyethylene-23 glycerol laurate, polyoxy-ethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxy-ethylene-6 tridecyl ether, laureth-2, laureth-3, laureth-4, PEG-3 castor oil, PEG 600 dioleate, PEG 400 dioleate, and mixtures thereof. Commercially available nonionic surfactants may include the SURFYNOL® range of acetylenic diol surfactants available from Air Products and Chemicals of Allentown, Pa. and the TWEEN® range of polyoxyethylene surfactants available from Fischer Scientific of Pittsburgh, Pa.

A binder may also be employed to facilitate the immobilization of the indicator on the wound suture. For example, water-soluble organic polymers may be employed as binders, such as polysaccharides and derivatives thereof. Polysaccharides are polymers containing repeated carbohydrate units, which may be cationic, anionic, nonionic, and/or amphoteric. In one particular embodiment, the polysaccharide is a nonionic, cationic, anionic, and/or amphoteric cellulosic ether. Suitable nonionic cellulosic ethers may include, but are not limited to, alkyl cellulose ethers, such as methyl cellulose and ethyl cellulose; hydroxyalkyl cellulose ethers, such as hydroxyethyl cellulose, hydroxypropyl cellulose, hydroxypropyl hydroxybutyl cellulose, hydroxyethyl hydroxypropyl cellulose, hydroxyethyl hydroxybutyl cellulose and hydroxyethyl hydroxypropyl hydroxybutyl cellulose; alkyl hydroxyalkyl cellulose ethers, such as methyl hydroxyethyl cellulose, methyl hydroxypropyl cellulose, ethyl hydroxyethyl cellulose, ethyl hydroxypropyl cellulose, methyl ethyl hydroxyethyl cellulose and methyl ethyl hydroxypropyl cellulose; and so forth.

The exact quantity of the indicator employed in the present invention may vary based on a variety of factors, including the sensitivity of the indicator, the presence of other additives, the desired degree of detectability (e.g., with an unaided eye), the suspected concentration of the microorganism, etc. In some cases, it is desirable to only detect the presence of bacteria at a pathogenic concentration. For example, a bacterial concentration of about 1×10³ colony forming units (“CFU”) per milliliter of growth media or more, in some embodiments about 1×10⁵ CFU/ml or more, in some embodiments about 1×10⁶ CFU/ml or more, and in some embodiments, about 1×10⁷ CFU/ml or more may be considered pathogenic. It should be understood that such concentrations may correlate to a liquid sample or a non-liquid sample (e.g., skin or obtained from skin) that is cultured in a growth media. Regardless, the indicator may be employed in an amount sufficient to undergo a detectable color change in the presence of bacteria at a desired concentration. For instance, the indicator may be present in a solution in an amount from about 1 wt. % to about 50 wt. %, in some embodiments from about 5 wt. % to about 40 wt. %, and in some embodiments, from about 10 wt. % to about 35 wt. %. Upon application, the indicator may constitute from about 0.01 wt. % to about 5 wt. %, in some embodiments from about 0.05 wt. % to about 3 wt. %, and in some embodiments from about 0.1 wt. % to about 1 wt. %, based on the dry weight of the wound suture.

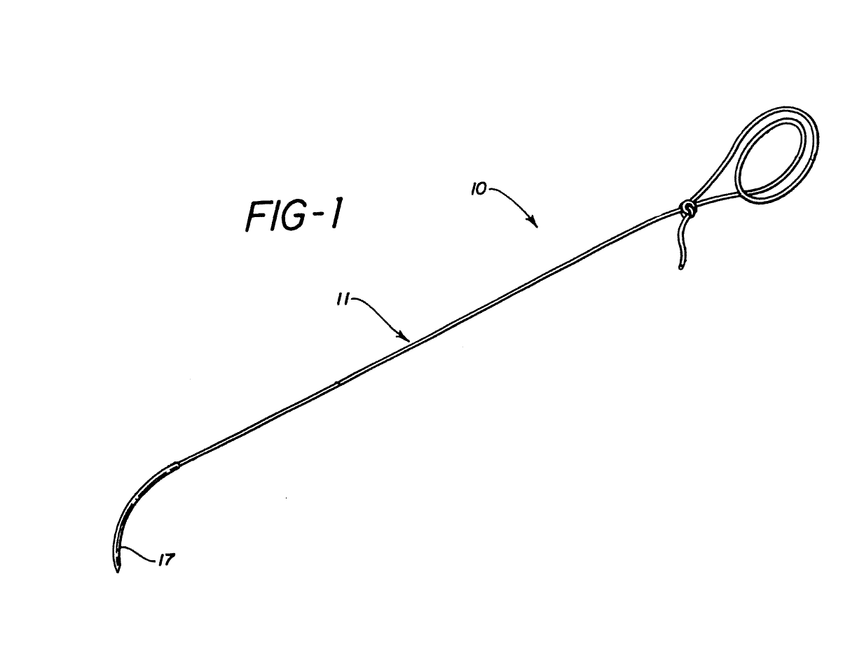

During a surgical procedure, the wound suture is typically attached to a needle as is well known in the art. Referring to FIG. 1, for example, a suture needle combination 10 is illustrated that contains a suture 11 attached to a surgical needle 17 using any conventional swaging technique. A user may grasp the needle 17 using an appropriate surgical instrument and penetrate bodily tissue at or near an incisional wound. The needle 17 may then be passed through the tissue until it penetrates the other side of the incisional wound. Once the needle 17 is passed through both sides of the incisional site, it may be pulled away from the tissue until the slip knot contacts the tissue at the point of entry of the needle. At this point, the needle 17 may be passed through each of the loops and knotted as is known in the art.

During or after closure of the wound with the suture, the presence of bacteria at the wound site may be readily detected by observing (either visually or with instrumentation) the color change. This allows detection of infection directly within the wound site, on the dermis and subcutaneous area of a wound site, or in exudates seeping from the suture holes and/or when the suture is removed. The color change may be rapid and may be detected within a relatively short period of time. For example, the change may occur in about 5 minutes or less, in some embodiments about 1 minute or less, in some embodiments about 30 seconds or less, in some embodiments about 20 seconds or less, and in some embodiments, from about 10 seconds to about 2 minutes. Conversely, the color change could be used to monitor the build up of microbial contamination on the skin surface over time. The microbes could be already present, in the or on the skin, in very small amounts and with time multiply to form a colony with sufficient number that a serious infection would result. They could also come from contamination after surgery through contact with infected hands, instruments or needles etc. Thus, the color change may indicate an instant contamination of a high number of microbes present or the build-up of microbes on or in the skin over time.

Regardless of when it occurs, the extent of the color change may be determined either visually or using instrumentation (e.g., optical reader) and provide a “real-time” indication of infection at a wound or incision site. The extent of the color change may be represented by a certain change in the absorbance reading as measured using a conventional test known as “CIELAB”, which is discussed in Pocket Guide to Digital Printing by F. Cost, Delmar Publishers, Albany, N.Y. ISBN 0-8273-7592-1 at pages 144 and 145. This method defines three variables, L*, a*, and b*, which correspond to three characteristics of a perceived color based on the opponent theory of color perception. The three variables have the following meaning:

L*=Lightness (or luminosity), ranging from 0 to 100, where 0=dark and 100=light;

a*=Red/green axis, ranging approximately from −100 to 100; positive values are reddish and negative values are greenish; and

b*=Yellow/blue axis, ranging approximately from −100 to 100; positive values are yellowish and negative values are bluish.

Because CIELAB color space is somewhat visually uniform, a single number may be calculated that represents the difference between two colors as perceived by a human. This difference is termed ΔE and calculated by taking the square root of the sum of the squares of the three differences (ΔL*, Δa*, and Δb*) between the two colors. In CIELAB color space, each ΔE unit is approximately equal to a “just noticeable” difference between two colors. CIELAB is therefore a good measure for an objective device-independent color specification system that may be used as a reference color space for the purpose of color management and expression of changes in color. Using this test, color intensities (L*, a*, and b*) may thus be measured using, for instance, a handheld spectrophotometer from Minolta Co. Ltd. of Osaka, Japan (Model # CM2600d). This instrument utilizes the D/8 geometry conforming to CIE No. 15, ISO 7724/1, ASTME1164 and JIS Z8722-1982 (diffused illumination/8-degree viewing system. The D65 light reflected by the specimen surface at an angle of 8 degrees to the normal of the surface is received by the specimen-measuring optical system. Typically, the indicator undergoes a color change that is represented by a ΔE of about 2 or more, in some embodiments about 3 or more, and in some embodiments, from about 5 to about 50.

The present invention may be better understood with reference to the following examples.

### EXAMPLE 1

The following braided sutures were employed in this example: coated VICRYL (Ethicon, Inc.), which is made from Polyglactin 910 (poly(glycolide-co-trimethylene carbonate)) and has a length of 27 inches; POLYSORB (US Surgical Corp.), which is made from lactomer 9-1 (poly(lactide-co-glycolide) and has a length of 18 inches; and DEXON “S” beige (Sherwood Davis & Geck), which is made from polyglycolic acid TT-20 and has a length of 18 inches. Each of the braided sutures was soaked in an acetontrile solution containing Reichardt's dye (20% wt/wt) overnight at ambient temperature in a sealed container. The sutures were then removed and placed on a glass plate and allowed to air dry in a fumehood over the weekend. The dried sutures had a blue purple color. This coating was not removed when rubbed with a dry paper towel.

### EXAMPLE 2

The following monofilament sutures were employed in this example: coated MONOCRYL (Ethicon, Inc.), which is made from poliglecaprone 25 (a copolymer of glycolide and ε-caprolactone) and has a length of 27 inches; CHROMIC GUT (Ethicon, Inc.), which is made primarily from collagen and has a length of 27 inches; BIOSYN (US Surgical Corp.), which is made from (glycomer 631, poly(dioxanone)-polyglycolide-trimethylene carbonate) and has a length of 36 inches; and PLAIN GUT (US Surgical), which is made primarily from collagen and has a length of 30 inches. Each of the monofilament sutures was soaked in an acetontrile solution containing Reichardt's dye (20% wt/wt) overnight at ambient temperature in a sealed container. The sutures were then removed and placed on a glass plate and allowed to air dry in a fumehood over the weekend. The monofilament sutures had a purple color, although not as deep a color as the braided filament sutures of Example 1.

### EXAMPLE 3

Examples 1 and 2 were repeated with an isopropanol solution of Reichardt's dye (12% wt/wt).

### EXAMPLE 4

Four (4) turkey thighs (Kroger grocery store) were slit with a scalpel to make several 3″ long incisions. These wounds were then sewn up using the dye-coated sutures of Example 1 (coated VICRYL and POLYSORB) and Example 2 (MONOCRYL). The sutures had needles attached to the suture and a herring bone pattern technique was used. The wound site area with the sutures was treated with a suspension of 1×10⁵ CFU (colony forming units) of S. aureus and the color of the sutures observed. Within seconds, the purple color of the sutures was discharged to colorless where the sutures had contacted the S. aureus.

### EXAMPLE 5

Eight (8) chicken legs (Kroger grocery store) were slit with a scalpel to make several 3″ long incisions. These wounds were then sewn up using the dye-coated sutures of Example 1 (DEXON “S” beige and POLYSORB) and Example 2 (BIOSYN). The sutures had needles attached to the suture and a herring bone pattern technique was used. The wound site area with the sutures was treated with a suspension of 1×10⁵ CFU (colony forming units) of S. aureus and the color of the sutures observed. Within seconds, the purple color of the sutures was discharged to colorless where the sutures had contacted the S. aureus.

### EXAMPLE 6

A turkey thigh and chicken leg from Example 4 (POLYSORB) was placed into sealable bags and left at ambient temperature overnight. Another chicken leg from Example 5 (DEXON “S” beige ) was also placed in a sealable bag and placed in a refrigerator overnight. After 10 hours, the thigh and legs at ambient temperature were observed and the sutures had turned colorless, thereby indicating that it had spoiled. The refrigerated chicken leg, however, had no discharge of the suture coating color, thereby indicating that it had not spoiled.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A wound suture comprising at least one filament, wherein the filament contains a solvatochromatic indicator that undergoes a detectable color change in the presence of bacteria.

2. The wound suture of claim 1, wherein the indicator is zwitterionic.

3. The wound suture of claim 2, wherein the zwitterionic indicator includes a merocyanine dye.

4. The wound suture of claim 3, wherein the merocyanine dye has the following structure:

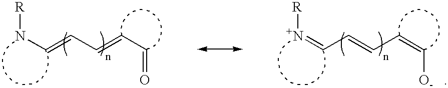

5. The wound suture of claim 2, wherein the zwitterionic indicator includes an N-phenolate betaine dye.

6. The wound suture of claim 5, wherein the N-phenolate betaine dye is Reichardt's dye.

7. The wound suture of claim 1, wherein the filament contains an absorbable polymer.

8. The wound suture of claim 1, wherein the filament has a multifilament configuration.

9. The wound suture of claim 1, wherein the filament has a monofilament configuration.

10. The wound suture of claim 1, wherein the indicator constitutes from about 0.01 wt. % to about 5 wt. %, based on the dry weight of the filament.

11. The wound suture of claim 1, wherein the indicator is coated on a surface of the filament.

12. The wound suture of claim 1, wherein the filament is attached to a needle.

13. A method for detecting the presence of bacteria at a surgical incision site, the method comprising:
passing a needled suture through tissue to create a wound closure, the suture containing a solvatochromatic indicator that undergoes a detectable color change in the presence of bacteria; and
thereafter, observing the suture for the color change.

14. The method of claim 13, wherein the indicator is zwitterionic.

15. The method of claim 14, wherein the zwitterionic indicator includes a merocyanine dye.

16. The method of claim 14, wherein the zwitterionic indicator includes an N-phenolate betaine dye.

17. The method of claim 16, wherein the N-phenolate betaine dye is Reichardt's dye.

18. The method of claim 13, wherein the suture contains a filament.

19. The method of claim 18, wherein the filament contains an absorbable polymer.

20. The method of claim 18, wherein the indicator is coated on a surface of the filament.

21. The method of claim 13, wherein the color change is correlated to the presence of bacteria at a concentration of about 1×10³ or more colony forming units per milliliter of a sample.

22. The method of claim 13, wherein the color change is visually observed.

23. The method of claim 13, wherein the indicator produces a visually observable spectral response in the presence of Streptococcus pyogenes, Pseudomonas aeruginosa, Enterococcus faecalis, Proteus mirabilis, Serratia marcescens, Enterobacter clocae, Acetinobacter anitratus, Klebsiella pneumoniae, Escherichia coli, Staphyloccus aureus, coagulase-negative Staphylococci, Enterococcus spp., or a combination thereof.
